(19) United States
(12) Patent Application Publication
(10) Pub. No.: US 2025/0319003 A1
(43) Pub. Date: Oct. 16, 2025

(54) MINERAL SUNSCREEN COMPOSITIONS

(71) Applicant: The Procter & Gamble Company, Cincinnati, OH (US)

(72) Inventors: Joseph Harry Jansen, Harrison, OH (US); Monica Salguero Marlow, Mason, OH (US); Daniel Lawrence Custer, West Chester, OH (US); Andrew Kevin Coyle, Centerville, OH (US); Delaney Brian Elliott, Cincinnati, OH (US); Scott Brian Bintrim, Carmel, IN (US)

(21) Appl. No.: 19/175,152

(22) Filed: Apr. 10, 2025

Related U.S. Application Data
(60) Provisional application No. 63/633,388, filed on Apr. 12, 2024.

(51) Int. Cl. A61K 8/35; A61K 8/27; A61K 8/34; A61Q 17/04

(52) U.S. Cl. CPC A61K 8/35; A61K 8/27; A61K 8/345; A61Q 17/04; A61K 2800/30; A61K 2800/524; A61K 2800/651

(57) ABSTRACT

A stable skin care composition that includes zinc oxide, a preservative system, and a dermatologically acceptable carrier. The preservative system includes a registered preservative material chosen from phenoxyethanol, benzyl alcohol, chlorphenesin, or mixtures thereof and optionally a booster comprising 1,2-octanediol and/or hydroxyacetophenone. The zinc oxide is dispersed throughout the skin care composition and the composition delivers a log reduction of ≥2 for yeast and mold, according to the Microbial Susceptibility Testing.

### FIELD OF THE INVENTION

The present disclosure generally relates to a physically and microbially stable mineral sunscreen composition. More particularly, the disclosure relates to a physically and microbially stable sunscreen composition that includes zinc oxide and at least 0.3% of a preservative system that includes a registered preservative, such as phenoxyethanol, chlorphenesin, and/or benzyl alcohol, and optionally a booster, such as 1,2-octanediol and/or hydroxyacetophenone.

### BACKGROUND OF THE INVENTION

Sunscreen is important for protecting the skin from harmful ultraviolet (UV) radiation, which can cause premature aging, sunburn, and an increased risk of skin cancer. When applied specifically to the face, sunscreen helps shield delicate facial skin from UV damage, which can reduce the appearance of wrinkles, fine lines, and age spots, while also promoting a healthier complexion. Some consumers may prefer mineral sunscreens over chemical sunscreens because they are perceived as gentler and less irritating. Additionally, mineral sunscreens provide a physical barrier on the skin's surface, reflecting and scattering UV rays, which some users perceive as a more natural and effective method for sun protection compared to the chemical absorption of UV rays by chemical sunscreen actives.

Zinc oxide (ZnO) is widely used as an active ingredient in mineral sunscreens, offering broad-spectrum protection against UVA and UVB rays. However, achieving its desired efficacy requires a relatively high concentration (e.g., ≥15% or ≥20%), posing challenges in manufacturing and application aesthetics due to the difficulties of dispersing ZnO particles within an oil-in-water emulsion. If the ZnO is not evenly dispersed, then it can impact the efficacy and/or can leave a white cast on the skin when it is applied, which can be undesirable for many users who prefer a more transparent application. Another issue is that syneresis has been observed in emulsions containing ZnO, where the formulation may separate or “weep” over time, resulting in the release of liquid, which can impact the stability, texture, and overall performance of the sunscreen.

Preserving sunscreens with ZnO against yeast and mold can also be challenging. There are many common preservatives for skin care products that act as cell wall disrupters, which work by interfering with the integrity and structure of microbial cell walls, leading to their disruption and subsequent inhibition of microbial growth and proliferation. However, the same mechanism that kills microorganisms can destabilize the emulsion and/or ZnO dispersion, posing additional difficulties in maintaining the physical stability of the sunscreen.

Therefore, there is a need for a physically stable oil-in-water sunscreen composition that is delivered with favorable aesthetics while containing an effective amount of ZnO dispersed throughout with a preservative system effective at delivering yeast/mold hostility.

### SUMMARY OF THE INVENTION

A skin care composition comprising: (a) zinc oxide; (b) from about 0.55% to about 2%, by weight of the composition, a preservative system comprising: (i) at least 0.15%, by weight of the composition, 1,2-octanediol; (ii) at least 0.5%, by weight of the composition, benzyl alcohol and/or at least 0.6%, by weight of the composition, phenoxyethanol; (c) a dermatologically acceptable carrier.

A skin care composition comprising: (a) zinc oxide; wherein the zinc oxide is dispersed; (b) from about 0.6% to about 1.5%, by weight of the composition, of a preservative system comprising: (i) a registered preservative material chosen from phenoxyethanol, benzyl alcohol, chlorphenesin, or mixtures thereof; (ii) optionally a booster chosen from 1,2-octanediol, hydroxyacetophenone, or mixtures thereof; (c) a dermatologically acceptable carrier, wherein the composition comprises a log reduction of ≥2 for yeast and mold, according to the Microbial Susceptibility Testing; wherein the skin care composition is an emulsion and the emulsion is stable.

A skin care composition comprising: (a) zinc oxide; (b) a preservative system comprising: (i) at least 0.2%, by weight of the composition, hydroxyacetophenone; (ii) at least 0.4%, by weight of the composition, phenoxyethanol and/or at least 0.2%, by weight of the composition, benzyl alcohol.

### BRIEF DESCRIPTION OF THE DRAWINGS

The patent or application file contains at least one drawing executed in color. Copies of this patent or patent application publication with color drawings will be provided by the Office upon request and payment of the necessary fee.

While the specification concludes with claims particularly pointing out and distinctly claiming the subject matter of the present invention, it is believed that the invention can be more readily understood from the following description taken in connection with the accompanying drawings, in which:

FIG. 1 shows the results from the Drawdown Method for Examples 11 and 13;

FIG. 2 shows the results from the Drawdown Method for Examples 6 and 8;

FIG. 3 shows the results from the Drawdown Method for Examples 12 and 14;

FIG. 4 shows the results from the Drawdown Method for Examples 7 and 9;

FIG. 5 shows the results from the Drawdown Method for Example 15 and 16;

FIG. 6A is a Scanning Electron Microscope (SEM) photograph of Example 11 taken at 5000× magnification;

FIG. 6B is energy dispersion spectroscopy photograph showing zinc dispersion of Example 11;

FIG. 7A is a Scanning Electron Microscope (SEM) photograph of Example 13 taken at 5000× magnification; and

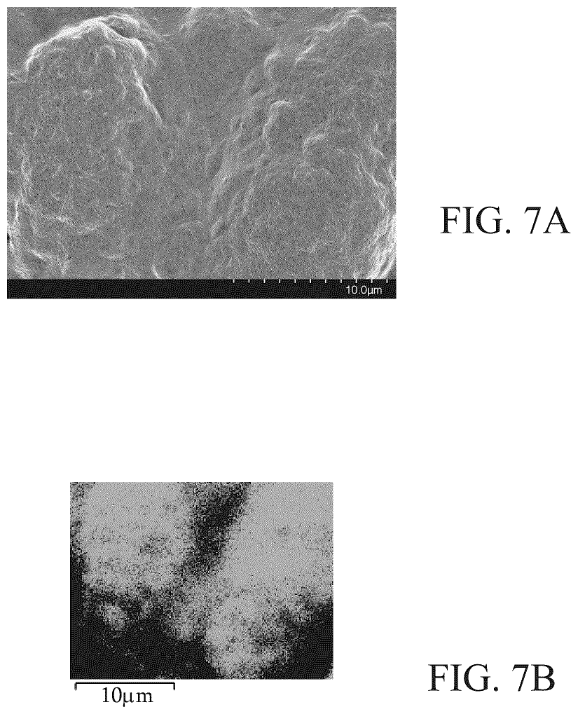

FIG. 7B
is energy dispersion spectroscopy photograph showing zinc dispersion of Example 13.

### DETAILED DESCRIPTION OF THE INVENTION

Sunscreen is crucial for protecting the skin from harmful UV radiation, which can cause various skin issues and increase the risk of skin cancer. Some people prefer mineral sunscreens and ZnO is a frequently used active ingredient in mineral sunscreens. However, it can be difficult to disperse ZnO and if ZnO is not evenly dispersed, the composition can become physically unstable, have an undesirable gritty texture, and/or have a white cast when spread on skin. Furthermore, it can be difficult for compositions that contain an effective amount of ZnO to meet the preservation requirements for yeast and mold, as established by European Pharmacopoeia <5.1.3> and the Microbial Susceptibility Testing (MST), described hereafter. Common preservative systems used to protect against microbial growth in skin care products can disrupt the sunscreen's emulsion and ZnO dispersion. Therefore, there is a need for a physically stable sunscreen composition with effective ZnO dispersion and preservation.

To create a physically stable sunscreen with an effective ZnO dispersion and an effective preservation system, it was first necessary to identify a preservation system that includes a registered preservative and optionally a booster, that could effectively inhibit and/or reduce microbial growth according to MST, described hereafter, and maintain the physical stability of the emulsion containing the dispersed ZnO. In response to some consumers demands for products that align with natural and clean beauty standards, it was also advantageous to avoid some preservatives (e.g., methylchloroisothiazolinone and methylisothiazolinone and DMDM hydantoin) to meet or exceed criteria like the “Clean at Sephora” requirements (see Best Clean Beauty Products 2022. Sephora. Retrieved Apr. 11, 2024, from www.sephora.com/beauty/clean-beauty-products, “Clean at Sephora” is an initiative in which the beauty retailer badged over 2,000 products as “clean,” in this case meaning free of ingredients like sulfates, parabens, formaldehyde, phthalates, and mineral oil).

Preservative systems were screened to see which ones had the potential to effectively reduce microbial concentrations. To provide microbial efficacy, the preservative system needs to be present in the water phase of the emulsion, since that is where the microbes reside. Therefore, preservative systems were screened to understand how the materials partitioned in the chassis (i.e., what percentage of the added material was delivered into the water phase of the chassis). The results from the screening are found in Table 1, below.

In Table 1, the Mean C_aq is the concentration of the preservative system material in the aqueous phase. Estimates of C_aq come from a model of solute partitioning in the composition. The model is trained from the measured C_aq of a training set of materials dissolved into the composition, Chassis A (see Table 3). The material properties of this training set are used as inputs to the model for predicting materials outside the training set, e.g. preservatives. C_aq of the training set was measured indirectly by gas chromatography mass spectrometry (GCMS) of product headspace, which is proportional to C_aq. The training set was comprised of volatile materials, but the model enables prediction of C_aq partitioning behavior for non-volatile preservatives, the partitioning of which is otherwise difficult to measure directly. The model is used to determine the Mean C_aq, which is the portion of preservative system material (includes both registered preservative materials and boosters) that is in the water phase. This calculation assumes that 0.25% w/w of the specific preservative system material was added to Chassis A. The Mean C_aq can also be expressed as % in water phase, as shown in Table 1. This value represents the Mean C_aq (concentration in aqueous phase) divided by the total % w/w added to the formula (0.25% w/w).

Preservative system materials that could be effective had a Mean C_aq greater than 0.05, indicating that at least some of the preservative material was in the water phase. Stronger preservative candidates generally had an even higher Mean C_aq (e.g., ≥0.08, ≥0.10, ≥0.13, ≥0.15, ≥0.20, ≥0.24, or ≥0.25).

As shown in Table 1, benzyl alcohol, phenoxyethanol, hydroxyacetophenone, 1,2-octanediol and 1,2-hexanediol all have a Mean c_aq ≥0.08 and were potential preservative system materials for the skin care emulsion with dispersed ZnO. Benzoic acid and salicylic acid also had a Mean C_aq that indicates it could be an effective preservative material; however, if it is used in an amount to reduce and/or inhibit microbial growth, it could lower the pH of the composition to <7. If the pH of the product is lowered below 7, the ZnO can form divalent Zn ions. These ions have the potential to enter the water phase of the product, which can result in product instability. This instability may lead to the degradation of the SPF (sun protection factor) agent present in the product.

Table 1 also shows that phenylpropanol, ethylhexylglycerin, methylheptylglycerin, and iodopropynyl butyl carbamate are likely not effective preservatives for this system because there is not a significant amount of the preservative in the water phase. Furthermore, methylparaben, propylparaben, methylchloroisothiazolinone and methylisothiazolinone, and DMDM hydantoin, may be effective preservatives, however, they are not preferred by consumers who are seeking beauty products that align with current natural and clean beauty standards and therefore were not explored further.

TABLE 1

| Preservative | Mean | % w/w in | SD of Mean |
|---|---|---|---|
| System Material | C_aq | Water Phase | C_aq |
| Benzyl Alcohol | 0.20 | 80% | 0.03 |
| Phenoxyethanol | 0.13 | 52% | 0.04 |
| Methylparaben | 0.28 | 112% | 0.03 |
| Propylparaben | 0.15 | 60% | 0.06 |
| Methylchloroisothiazolinone | 0.28 | 112% | 0.02 |
| and Methylisothiazolinone | | | |
| Benzoic Acid | 0.25 | 100% | 0.03 |
| Hydroxyacetophenone | 0.25 | 100% | 0.03 |
| DMDM Hydantoin | 0.31 | 124% | 0.00 |
| 1,2-Octanediol | 0.08 | 32% | 0.03 |
| 1,2-Hexanediol | 0.25 | 100% | 0.03 |
| Salicylic Acid | 0.24 | 96% | 0.05 |
| Phenylpropanol | 0.05 | 20% | 0.02 |
| Ethylhexylglycerin | 0.01 | 4% | 0.01 |
| Methylheptylglycerin | 0.01 | 4% | 0.01 |
| Iodopropynyl Butyl Carbamate | 0.00 | 0% | 0.00 |

Next, the preservative system materials that were identified as potential candidates in Table 1, above, were tested in various combinations and at various levels to see if they would be effective against yeast and mold (Y/M) in the Microbial Susceptibility Testing, described hereafter. The results from this experiment are shown in Table 2, below. A sample passes MST if, at any point during the shelf life, there is a reduction of fungal microbes by 2 logs or greater (rounded to the nearest integer) by 14 days post-inoculation. To test if the preservative system could be effective across the shelf life of the product, the product was tested under accelerated stability conditions. The preservative system was considered potentially effective for the shelf life of the product if it had at least a 2-log reduction rounded to the nearest integer when the sample is inoculated per MST at the following points: (1) soon after the sample is made (Initial Day 14 Y/M); (2) after 1 month at 40° C. (1 Month at 40° C. Day 14 Y/M); and (3) 3 months at 40° C. (3 Months at 40° C. Day 14 Y/M), according to MST, described hereafter.

As shown in Table 2, below, all preservative combinations, except for the one containing 0.4875% Benzyl Alcohol and 0.68% 1,2-Hexendiol (and) 1,2-Octanediol (containing 0.17%-0.34% 1,2-Octanediol), had at least a 2-log reduction at all three accelerated stability time points. This indicates that these preservative systems have the potential to be effective for preserving sunscreen emulsions that contain ZnO.

TABLE 2

| | Initial | 1 Month at 40° C. | 3 Months at 40° C. |
|---|---|---|---|
| | Day 14 Y/M | Day 14 Y/M | Day 14 Y/M |
| Preservative System | (Log Reduction) | (Log Reduction) | (Log Reduction) |
| 0.475% Phenoxyethanol & | 3.4 | 3.0 | 3.2 |
| 0.3% Hydroxyacetophenone¹ | | | |
| 0.39% Benzyl Alcohol & | 3.1 | 3.1 | 2.9 |
| 0.3% Hydroxyacetophenone¹ | | | |
| 0.5625% Phenoxyethanol & | 2.2 | 2.1 | 1.9 |
| 0.68% 1,2-Hexanediol (and) | | | |
| 1,2-Octanediol² | | | |
| 0.7125% Phenoxyethanol & | 4.0 | 3.4 | 3.1 |
| 0.76% 1,2-Hexanediol (and) | | | |
| 1,2-Octanediol² | | | |
| 0.4875% Benzyl Alcohol & | 2.2 | 1.9 | 1.1 |
| 0.68% 1,2-Hexanediol (and) | | | |
| 1,2-Octanediol² | | | |
| 0.585% Benzyl Alcohol & | 4.0 | 3.3 | 3.1 |
| 0.76% 1,2-Hexanediol (and) | | | |
| 1,2-Octanediol² | | | |
| Nil Preservative | <1.1 | N/A | N/A |

¹SymSave ® H from Symrise ®
²SymDiol ® 68 from Symrise ®

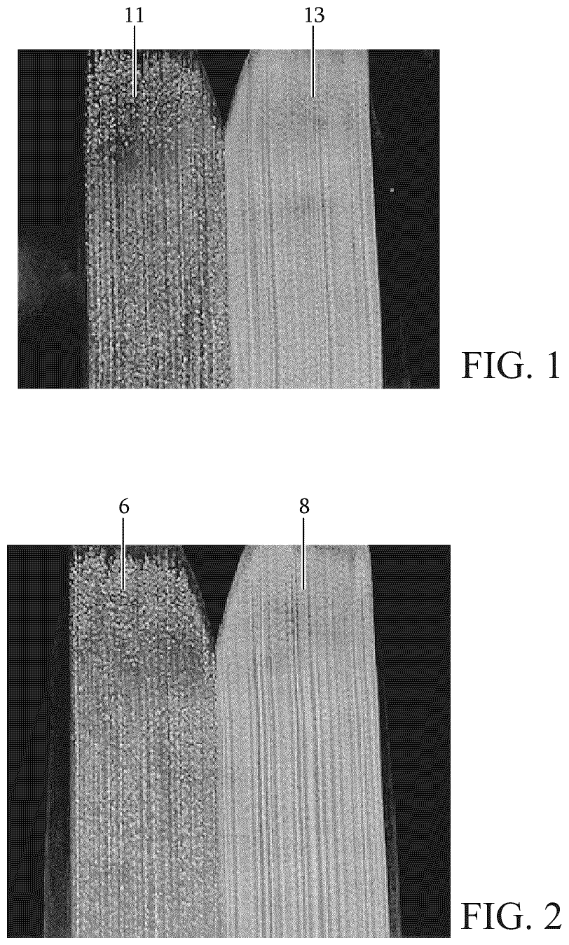

Finally, example compositions that included ZnO and a preservative system were tested to assess the quality of the ZnO dispersion. If the ZnO is well dispersed, the composition generally has better physical stability and consumer preferred aesthetic properties. The composition has a uniform dispersion that is likely consumer acceptable if it forms a smooth uniform film during the Drawdown Method. The Drawdown Method was performed as follows:
1. Samples are mixed in a Max 60 container at 2000 RPMs for 2 minutes in a Hauschild Speedmixer, model DAC600.1 FVZ.
2. The high-speed mixed sample is dispensed as a 1.6 g bead towards the edge on the black portion of a Leneta Form 2A Opacity Chart. In this instance, two beads of two different samples were dispensed onto each card for easier qualitative comparison.
3. Using a 1.5 mil Bird Film Applicator®, the product beads were spread down the card, resulting in a pattern resembling FIGS. 1-5.
4. Film properties were then qualitatively reported 1-3 hours after completing the drawdowns. A composition passed the Drawdown Method if by visual detection the drawdown appears as a smooth, uniform film, which can indicate good ZnO dispersion. As used herein, “visual detection” means that a human viewer can visually discern if the product appears grainy, clumpy, inconsistent, or otherwise separated with the unaided eye (except for standard corrective lenses adapted to compensate for near-sightedness, farsightedness, or astigmatism, or other corrected vision) in lighting at least equal to the illumination of a standard 100-watt incandescent white light bulb at 30 cm.

FIG. 1 shows the results from the Drawdown Method for drawdown 11, which uses a bead of Example 11 (see Table 5, below), and drawdown 13, which uses a bead of Example 13 (see Table 6, below). Both examples 11 and 13 include ˜20% ZnO. Example 11 has a preservative system that includes 0.15% hydroxyacetophenone and 0.375% phenoxyethanol and Example 13 has a preservative system that includes 0.3% hydroxyacetophenone and 0.475% phenoxyethanol. Drawdown 11 appears inconsistent and grainy, indicating that the ZnO is not well dispersed, and this composition may not be consumer preferred. Drawdown 13 has a better dispersion, as indicated by the smooth, uniform appearance of the drawdown. It was surprising that a composition with more preservative would have better ZnO dispersion because hydroxyacetophenone and phenoxyethanol are both cell wall disrupters and cell wall disrupters are known to destabilize the emulsion and/or the ZnO dispersion.

FIG. 2 shows the results from the Drawdown Method for drawdown 6, which uses a bead of Example 6 (see Table 4, below), and drawdown 8, which uses a bead of Example 8 (see Table 5, below). Both examples 6 and 8 include ˜20% ZnO. Example 6 has a preservative system that includes 0.68% 1,2-hexanediol (and) 1,2-octanediol and 0.5625% phenoxyethanol and Example 8 has a preservative system that includes 0.76% 1,2-hexanediol (and) 1,2-octanediol and 0.7125% phenoxyethanol. Drawdown 6 appears grainy, indicating that the ZnO is not well dispersed, and this composition may not be consumer preferred. Drawdown 8 has a better dispersion, as indicated by the smooth, uniform appearance of the drawdown. Again, it was surprising that a composition with more preservative would have better ZnO dispersion because 1,2-Octanediol and phenoxyethanol are known to destabilize the emulsion and/or the ZnO dispersion.

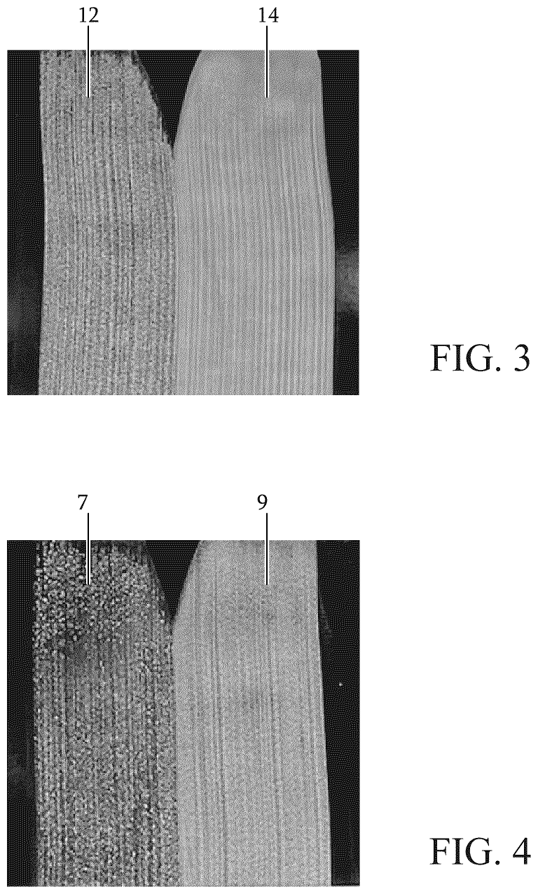

FIG. 3 shows the results from the Drawdown Method for drawdown 12 that uses a bead of Example 12 (see Table 6, below) and drawdown 14 that uses a bead of Example 14 (see Table 6, below). Both examples 12 and 14 include ˜20% ZnO. Example 12 has a preservative system that includes 0.15% hydroxyacetophenone and 0.325% benzyl alcohol, and Example 14 has a preservative system that includes 0.3% hydroxyacetophenone and 0.39% Benzyl Alcohol. Drawdown 12 appears grainy, indicating that the ZnO is not well dispersed, and this composition may not be consumer preferred. Drawdown 14 has a better dispersion, as indicated by the smooth, uniform appearance of the drawdown. Again, it was surprising that a composition with more preservative would have better ZnO dispersion.

FIG. 4 shows the results from the Drawdown Method for drawdown 7 that uses a bead of Example 7 (see Table 5, below) and drawdown 9 that uses a bead of Example 9 (see Table 5, below). Both examples 7 and 9 include ˜20% ZnO. Example 7 has a preservative system that includes 0.68% 1,2-hexanediol (and) 1,2-octanediol and 0.4875% benzyl alcohol, and Example 9 has a preservative system that includes 0.76% 1,2-hexanediol (and) 1,2-Octanediol and 0.585% Benzyl Alcohol. Drawdown 7 appears grainy, indicating that the ZnO is not well dispersed, and this composition may not be consumer preferred. Drawdown 9 has a better dispersion, as indicated by the smooth, uniform appearance of the drawdown. Again, it was surprising that a composition with more preservative would have better ZnO dispersion.

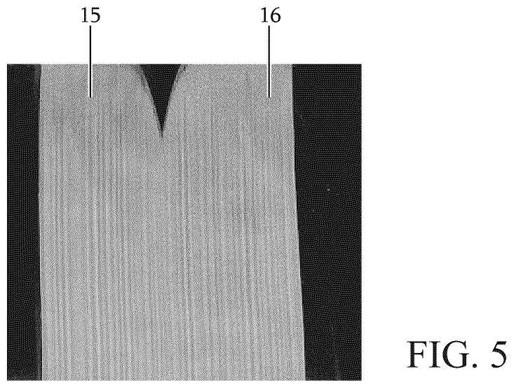

FIG. 5 shows the results from the Drawdown Method for drawdown 15 that uses a bead of Example 15 (see Table 6, below) and drawdown 16 that uses a bead of Example 16 (see Table 6, below). Both examples 15 and 16 include ˜20% ZnO. Example 15 has a preservative system that includes 0.6% phenoxyethanol (and) chlorphenesin (and) aqua (and) glycerin, and Example 16 has a preservative system that includes 1% phenoxyethanol (and) chlorphenesin (and) aqua (and) glycerin. While both drawdowns had good ZnO dispersions indicated by the smooth, uniform appearance of the drawdown, Example 15 did not meet the 2-log reduction according to the MST protocol, as shown in Table 6.

### Composition

The skin care composition can be a homogenous emulsion that includes a continuous phase and a dispersed phase. The continuous phase can include a dermatologically acceptable carrier, which can typically include water, at least a portion of the preservative system, and water soluble skin care actives. The dispersed phase can include oils, and the ZnO and other mineral sunscreen components.

The dermatologically acceptable carrier enables other components (e.g., actives) to be delivered to the skin at an appropriate concentration. The carrier can thus act as a diluent, dispersant, solvent, or the like for particulate material, which helps ensure that it can be applied to and distributed evenly over the selected target at an appropriate concentration. The carrier may contain one or more dermatologically acceptable solid, semi-solid or liquid fillers, diluents, solvents, extenders and the like. The carrier may be solid, semi-solid, or liquid. In some instances, the carrier can be inert, or it can provide benefits of its own to keratinous tissue. Concentrations of the carrier can vary with the carrier selected and the intended concentrations of the composition components.

The skin care composition can be free of or formulated without formaldehyde and formaldehyde-releasing agents. The skin care composition can be free of DMDM hydantoin, imadazolidinyl urea, diazolidinyl urea, sodium hydroxyl, methyl glycinate, and combinations thereof. The skin care composition can also be free of parabens and/or sulfate-based surfactants including SLS and SLES.

The composition can be a physically stable emulsion. A composition is physically stable if by visual detection there is no phase separation. As used herein, “visual detection” means that a human viewer can visually discern if the product had separated into 2 distinct layers in a 2 oz clear glass jar with the unaided eye (except for standard corrective lenses adapted to compensate for near-sightedness, farsightedness, or astigmatism, or other corrected vision) in lighting at least equal to the illumination of a standard 100-watt incandescent white light bulb at 30 cm.

The pH of the composition can be >7, alternatively the pH can be from about 7.1 to about 8.2 according to the pH Test Method, described herein.

The composition can be an “SPF” product or a “Broad Spectrum SPF” product with an SPF ≥15, alternatively ≥20, alternatively ≥25, alternatively ≥30, alternatively ≥45, and alternatively ≥50, according to the broad spectrum test in Over-the-Counter Monograph M020: Sunscreen Drug Products for Over-the-Counter Human Use, § M020.90 (Sep. 24, 2021).

The composition can have a limited number of ingredients, in particular a limited number of ingredients that need to be reported on the INCI (International Nomenclature Cosmetic Ingredient) statement on the package. The composition can ≤25 ingredients, alternatively ≤20 ingredients, alternatively ≤15 ingredients, and alternatively ≤12 ingredients.

The weight ratio of zinc oxide to preservative system can be from about 10:1 to about 40:1, alternatively from about 15:1 to about 35:1, alternatively from about 18:1 to about 30:1, alternatively from about 18:1 to about 26:1, and alternatively from about 20:1 to about 25:1.

### Preservative System

A preservative system is added to a composition to prevent decomposition by microbial growth and/or by undesirable chemical changes. Additionally, preservative systems are designed to protect consumers against potential, inadvertent, low-level microbiological contamination during consumer use through the product expiration date. The preservative system can include registered preservative materials and optionally booster materials.

The composition can include >0.25% preservative system, alternatively ≥0.35%, alternatively ≥0.55%, alternatively ≥0.6%, alternatively ≥0.65%, alternatively ≥0.7%, alternatively ≥0.75%, alternatively ≥0.77%, alternatively ≥0.8%, alternatively ≥0.9%, alternatively ≥1%. The composition can include ≤1.6% preservative system, alternatively ≤1.5%, alternatively ≤1.4%, alternatively ≤1.3%, alternatively ≤1.2%, and alternatively ≤1.1%.

The preservative system phase can be in the water phase of the emulsion and ≥30% of the preservative system can be water phase, alternatively ≥40%, alternatively ≥50%, alternatively ≥60%, alternatively ≥70%, alternatively ≥75%, and alternatively ≥80%.

The composition can include a preservative system chosen from benzyl alcohol, phenoxyethanol, hydroxyacetophenone, 1,2-octanediol, chlorphenesin, or mixtures thereof.

The composition can include ≥0.26% phenoxyethanol, alternatively ≥0.3%, alternatively ≥0.33%, alternatively ≥0.35%, alternatively ≥0.40%, alternatively ≥0.45%, alternatively ≥0.5%, alternatively ≥0.55%, alternatively ≥0.6%, alternatively ≥0.65%, and alternatively ≥0.70%.

The composition can include ≥0.5% benzyl alcohol, and alternatively ≥0.55%.

The composition can include ≥0.16% hydroxyacetophenone, alternatively ≥0.2%, alternatively ≥0.25%, alternatively ≥0.27%, and alternatively ≥0.30%.

The composition can include ≥0.15% 1,2-octanediol, alternatively ≥0.18%, alternatively ≥0.19%, alternatively ≥0.20%, alternatively ≥0.25%, alternatively ≥0.30%, alternatively ≥ 0.35%.

The composition can include ≥0.07% chlorphenesin, alternatively ≥0.1%, alternatively ≥0.12%, alternatively ≥0.15%, and alternatively ≥0.20%.

The preservative system can include one or more preservative materials and a booster. The preservative system can include 1,2-octanediol and benzyl alcohol. The composition can include from about 0.15% to about 0.5% 1,2-octanediol, alternatively from about 0.18% to about 0.4%, alternatively from about 0.18% to about 0.38% and the composition can include about 0.4% to about 1% benzyl alcohol, alternatively from about 0.45% to about 0.8%, alternatively from about 0.5% to about 0.75%, and alternatively from about 0.55% to about 0.7%. The weight ratio of benzyl alcohol to 1,2-octanediol can be from about 1.4:1 to about 4:1, alternatively from about 1.5:1 to about 3:1. The weight ratio of benzyl alcohol to 1,2-octanediol can be ≥1:1, alternatively ≥1.5:1.

The preservative system can include 1,2-octanediol and phenoxyethanol. The composition can include from about 0.15% to about to about 0.5% 1,2-octanediol, alternatively from about 0.18% to about 0.4%, alternatively from about 0.18% to about 0.38% and from about 0.6% to about 1% phenoxyethanol, alternatively from about 0.65% to about 0.9%, alternatively from about 0.67% to about 0.85%, and alternatively from about 0.7% to about 0.8%. The weight ratio of phenoxyethanol to 1,2-octanediol can be from about 1.45:1 to about 5:1, alternatively from about 1.5:1 to about 4:1, and alternatively from about 2:1 to about 3.5:1. The weight ratio of phenoxyethanol to 1,2-octanediol can be ≥1.5:1, alternatively ≥2:1, alternatively ≥2.5:1, alternatively ≥2.75:1, alternatively ≥2.9:1, and alternatively ≥3:1.

The composition can include two or more registered preservative materials. The preservative system can include hydroxyacetophenone and phenoxyethanol. The composition can include from about 0.2% to about 0.8% hydroxyacetophenone, alternatively from about 0.25% to about 0.6%, alternatively from about 0.27% to about 0.5%, and alternatively from about 0.3% to about 0.4% and the composition can include from about 0.2% to about 0.8% phenoxyethanol, alternatively from about 0.25% to about 0.75%, alternatively from about 0.3% to about 0.7%, alternatively from about 0.35% to about 0.65%, alternatively from about 0.4% to about 0.6%, alternatively from about 0.43% to about 0.55%, and alternatively from about 0.47% to about 0.53%.

The preservative system can include hydroxyacetophenone and benzyl alcohol. The composition can include from about 0.2% to about 0.8% hydroxyacetophenone, alternatively from about 0.25% to about 0.6%, alternatively from about 0.27% to about 0.5%, and alternatively from about 0.25% to about 0.4% and the composition can include from about 0.2% to about 0.6% benzyl alcohol, alternatively from about 0.3% to about 0.55%, alternatively from about 0.3% to about 0.5%, and alternatively from about 0.35% to about 0.45%.

The preservative system can include chlorphenesin and phenoxyethanol. The composition can include from about 0.07% to about 0.7% chlorphenesin, alternatively from about 0.1% to about 0.5%, alternatively from about 0.15% to about 0.4%, and alternatively from about 0.2% to about 0.3% and the composition can include from about 0.16% to about 1% phenoxyethanol, alternatively from about 0.2% to about 0.9%, alternatively from about 0.3% to about 0.8%, alternatively from about 0.4% to about 0.7%, alternatively from about 0.5% to about 0.6%.

The composition can be formulated without, free of, or substantially free of organic acids. The composition can be formulated without, free of, or substantially free of benzoic acid. Alternatively, the composition can contain ≤0.2% of an organic acid, alternatively ≤0.1%, and alternatively ≤0.05%.

The composition can be formulated without, free of, or substantially free of one or more of the following preservatives: methylchloroisothiazolinone and methylisothiazolinone (commercially available as Kathon® CG); DMDM hydantoin (commercially available as Glydant®); and/or DMDM hydantoin and iodopropynyl butylcarbamate (commercially available as Glydant® Plus Liquid).

The composition can be formulated without, free of, or substantially free of one or more of the following preservatives: phenylpropanol, ethylhexylglycerin, methylheptylglycerin, and/or iodopropynyl butyl carbamate because these are likely ineffective because very little is present in the water phase.

### Mineral UV Actives

The composition can contain one or more mineral UV actives including titanium dioxide and/or zinc oxide.

In some examples, zinc oxide is the only mineral UV active present and/or the only UV active present. The composition can be made with naturally derived non-nano zinc oxide. The composition can include from about 7% to about 25% zinc oxide, alternatively from about 9% to about 25%, alternatively from about 11% to about 25%, alternatively from about 12% to about 25%, alternatively from about 13% to about 25%, alternatively from about 17% to about 25%, alternatively from about 20% to about 25%, and alternatively from about 24% to about 25%. In some examples, the composition can have ≤25%, alternatively ≤22%, alternatively ≤20%, and alternatively ≤17%.

The composition can contain titanium dioxide. The composition can include from about 2% to about 25% titanium dioxide, alternatively from about 3% to about 20%, alternatively from abut 4% to about 18%, alternatively from about 5% to about 16%, alternatively from about 7% to about 15%, alternatively from about 8% to about 13%, and alternatively from about 9% to about 12%. The composition can include ≤25% titanium dioxide, alternatively ≤20%, alternatively ≤ 15%, alternatively ≤12%, and alternatively ≤10%.

The composition can be formulated without, substantially free of, or free of chemical sunscreen actives.

### Chemical Sunscreen Actives

The composition can also contain chemical sunscreen actives, which are substances that absorb harmful ultraviolet radiation. Chemical sunscreens can be classified, depending on the type of radiation they protect against, as either UV-A or UV-B chemical sunscreens. UV-A chemical sunscreen actives generally absorb radiation in the 320 to 400 nm region of the ultraviolet spectrum and UV-B chemical sunscreen actives generally absorb radiation in the 280 to 320 nm region of the ultraviolet spectrum.

Chemical sunscreen actives can include up to 15% aminobenzoic acid (PABA), up to 3% avobenzone, up to 3% cinoxate, up to 3% dioxybenzone, up to 4% ensulizole, up to 15% homosalate, up to 5% meradimatem up to 7.5% octinoxate, up to 5% octisalate, up to 10% octocrylene, up to 6% oxybenzone, up to 8% padimate, up to 10% sulisobenzone, up to 12% trolamine salicylate, or mixtures thereof.

### Emulsifier

The skin care composition can contain an emulsifier. The preferred emulsifier can promote the formation of the emulsion, stabilize the composition, and can be chemically and physically compatible with the other components of the composition.

The skin care composition can contain from about 0.10% to about 2%, alternatively from about 0.2% to about 1.5%, alternatively from about 0.4% to about 1.2%, and alternatively from about 0.6% to about 0.8% of an emulsifier. The skin care composition can contain less than 2%, alternatively less than 1.75%, alternatively less than 1.5%, alternatively less than 1.25%, alternatively less than 1%, and alternatively less than 0.8% of an emulsifier.

The emulsifier can be a non-silicone containing emulsifier. Non-limiting examples of non-silicone containing emulsifiers can include polyethylene glycol 20 sorbitan monolaurate (Polysorbate 20), polyethylene glycol 5 soya sterol, Steareth-20, Ceteareth-20, PPG-2 methyl glucose ether distearate, Ceteth-10, Polysorbate 80, cetyl phosphate, potassium cetyl phosphate, diethanolamine cetyl phosphate, Polysorbate 60, glyceryl stearate, polyoxyethylene 20 sorbitan trioleate (Polysorbate 85), sorbitan monolaurate, polyoxyethylene 4 lauryl ether sodium stearate, polyglyceryl-4 isostearate, hexyl laurate, PPG-2 methyl glucose ether distearate, PEG-100 stearate, cetearyl glucoside and mixtures thereof.

The emulsifier can be a fatty acid ester mixtures based on a mixture of sorbitan or sorbitol fatty acid ester and sucrose fatty acid ester, wherein the fatty acid in each case is preferably C₈-C₂₄, more preferably C₁₀-C₂₀. The preferred fatty acid ester emulsifier is a mixture of sorbitan or sorbitol C₁₆-C₂₀ fatty acid esters with sucrose C₁₀-C₁₆ fatty acid esters, especially sorbitan stearate and sucrose cocoate. This is commercially available from ICI under the trade name Arlatone 2121.

The skin care composition can include a silicone-containing emulsifier. A wide variety of silicone containing emulsifiers can be used. These silicone emulsifiers are typically organically modified organopolysiloxanes which are also known to those skilled in the art as silicone surfactants. Useful silicone emulsifiers include dimethicone copolyols. These materials are polydimethylsiloxanes which have been modified to contain polyether side chains such as polyethylene oxide chains, polypropylene oxide chains, mixtures of these chains, and polyether chains containing groups derived from both ethylene oxide and propylene oxide. Other examples include alkyl-modified dimethicone copolyols, that is, compounds containing pendant C₂-C₃₀ side chains.

Dimethicone copolyols can also be used as an emulsifiers herein including polydimethylsiloxane polyether copolymers with pendant polyethylene oxide sidechains, polydimethylsiloxane polyether copolymers with pendant polypropylene oxide sidechains, polydimethylsiloxane polyether copolymers with pendant mixed polyethylene oxide and polypropylene oxide sidechains, polydimethylsiloxane polyether copolymers with pendant mixed poly(ethylene) (propylene) oxide sidechains, polydimethylsiloxane polyether copolymers with pendant organobetaine sidechains, polydimethylsiloxane polyether copolymers with pendant carboxylate sidechains, polydimethylsiloxane polyether copolymers with pendant quaternary ammonium sidechains; and also further modifications of the preceding copolymers containing pendant C2-C30 straight, branched, or cyclic alkyl moieties. Examples of commercially available dimethicone copolyols useful herein sold by Dow Corning Corporation are Dow Corning® 190, 193, Q2-5220, 2501 Wax, 2-5324 fluid, and 3225C (this later material being sold as a mixture with cyclomethicone). Cetyl dimethicone copolyol is commercially available as a mixture with polyglyceryl-4 isostearate (and) hexyl laurate and is sold under the tradename ABIL® WE-09 (available from Goldschmidt). Cetyl dimethicone copolyol is also commercially available as a mixture with hexyl laurate (and) polyglyceryl-3 oleate (and) cetyl dimethicone and is sold under the tradename ABIL® WS-08 (also available from Goldschmidt). Other nonlimiting examples of dimethicone copolyols also include lauryl dimethicone copolyol, dimethicone copolyol acetate, dimethicone copolyol adipate, dimethicone copolyolamine, dimethicone copolyol behenate, dimethicone copolyol butyl ether, dimethicone copolyol hydroxy stearate, dimethicone copolyol isostearate, dimethicone copolyol laurate, dimethicone copolyol methyl ether, dimethicone copolyol phosphate, and dimethicone copolyol stearate. See International Cosmetic Ingredient Dictionary, Fifth Edition, 1993, which is incorporated by reference herein in its entirety.

In another example, the emulsifier can include cetearyl olivate and sorbitan olivate (commercially available as Olivem® 1000 from HallStar®) and/or polyglyceryl-3 cetearyl ether olivate (commercially available as Galolive® Bioten from Acme-Hardesty®). It is believed that other emulsifiers that include a mixture of waxy esters made from fatty alcohols and fatty acids derived from natural sources (e.g., olive, palm, corn, palm, rapeseed, soybean, sunflower, coconut, tallow, canola, castor) with a carbon chain length ranging from C12-C22 could be used in stable skin care compositions.

Additional examples of emulsifiers that can be used are found in U.S. Pat. No. 6,174,533, incorporated herein by reference.

### Polymer

The composition, in particular the water phase, can include one or more polymers. The polymer can help thicken the skin care composition, as well as help provide a smooth, silky feeling during and after application to the skin.

The composition may comprise greater than 0.2%, alternatively greater than 0.3%, alternatively greater than 0.4%, alternatively greater than 0.5%, alternatively greater than 0.6%, alternatively greater than 0.7%, or alternatively greater than or equal to 0.8% polymer. The composition may comprise less than or equal to 3%, alternatively less than or equal to 2.5%, alternatively less than or equal to 2%, alternatively less than or equal to 1.6%, alternatively less than or equal to 1.4%, alternatively less than or equal to 1.3%, alternatively less than or equal to 1.0% polymer.

The polymer can include an acrylate/taurate copolymer and in some examples the polymer can be a sodium acrylate/sodium acryloyldimethyl taurate copolymer. In some examples, the composition can include Sodium Acrylate/Sodium Acryloyldimethyl Taurate Copolymer & C15-19 Alkane & Polyglyceryl-6 Laurate & Polyglycerin-6 (commercially available as SEPILIFE™ NUDE from Seppic® Corporation). In some cases, the composition can include sodium acrylate/sodium acryloyldimethyl taurate copolymer & C15-19 alkane & Lauryl Glucoside (commercially available as Natursol™ EMI132 from SNF Floerger). In other examples, the composition can include sodium polyacryloyldimethyl taurate, hydroxyethyl acrylate/sodium acrloyldimethyl taurate copolymer, Sodium acrylate/Sodium Acryloyldimethyl Taurate copolymer, Ammonium Polyacryloyldimethyl taurate, Ammonium Acryloyldimethyltaurate/VP Copolymer, sodium acrylates copolymer, Polyacrylate Crosspolymer-11, Polyacrylate Crosspolymer-6, carbomer, Acrylates/C10-30 Alkyl Acrylate Crosspolymer, and combinations thereof.

The composition can include one or more of the following polymers: coconut alkanes (and) coco-caprylate/caprate, tocopherol, caprylic/capric triglyceride, cetearyl alcohol, coco-glucoside polyhydroxystearic acid, arachidyl glucoside, arachidyl alcohol, xanthan gum or mixtures thereof.

### Method of Use

The skin care composition may be applied to the face, neck, or any portion of the skin. The composition can be applied after washing to clean and/or dry skin. The skin care composition can be used as a moisturizer, or it can be used in addition to a separate moisturizing composition and can be applied after the moisturizer. The skin care composition can be applied during the day, before UV exposure and can be reapplied as needed.

The skin care composition can be applied under makeup and you can wait a few minutes for the skin care composition to soak in before applying makeup. The skin care composition can be invisible under makeup. The skin care composition can feel light and smooth on your skin. The skin care composition can have a matte appearance.

The composition can provide broad spectrum SPF 30. The composition can absorb quickly and does not leave a white cast across all skin tones and even body hair. The composition can be fragrance-free. The composition can be gentle and lightweight and safe for sensitive skin. The skin care composition does not have a tacky and/or greasy skin-feel.

### Definitions

As used herein, “clean” refers to cosmetic compositions that are formulated without the following ingredients: Acrylate Monomers, Aluminum Salts, Animal Musk's/Fats/Oils, Benzophenones and related compounds, Butoxyethanol, Butylated hydroxyanisole (BHA), Butylated hydroxytoluene (BHT), Carbon Black or Black 2, Coal Tar, Ethanolamines, Formaldehyde and Formaldehyde-releasing agents, Hydroquinone, Lead and Lead Acetate, Mercury and Mercury Compounds (Thimerisol), Methoxyethanol, Methyl Cellosolve, Methylchloroisothiazolinone and Methylisothiazolinone, Mineral Oil, Nanomaterials, Chemical sunscreen actives (including oxybenzone, avobenzone, and octinoxate), Parabens (including butyl-, methyl-, and propylparaben), Petrolatum and Paraffin, Phenoxyethanol, Phthalates, Plastic Microbeads, Polyacrylamide and Acrylamide, Resorcinol, Retinyl Palmitate, Styrene, Sulfates (including sodium lauryl sulfate and sodium laureth sulfate), Talc, Toluene, Triclosan and Triclocarban, Acetaldehyde, Acetone, Acetonitrile, Benzalkonium chloride, Bisphenol A (BPA), Ethylenediaminetetraacetic acid (EDTA), Methylene chloride, Polytetrafluoroethylene (PTFE), Perfluorooctanoic acid (PFOA), 1,4 Dioxane, Octinoxate, Nitromusks and Polycyclic Musks, PFAS compounds, Ethoxylated Ingredients, Polyethylene glycol (PEG), Cyclic Silicones, volatile linear silicones including silicone elastomers which contain volatile linear silicones, and derivatives and combinations thereof.

As used herein, “formulated without” means that the ingredient is not intentionally added. However, “formulated without” does not guarantee “100% free from” since trace contaminants are possible.

“Improve the appearance of” means providing a measurable, desirable change or benefit in skin appearance, which may be quantified, for example, by a decrease in redness, inflammation, and/or plaque scales.

As used herein, “molecular weight” refers to weight average molecular weight unless otherwise stated. Molecular weights are measured using the industry standard method of gel permeation chromatography (“GPC”).

As used herein, “skin care” means regulating and/or improving a skin condition. Some nonlimiting examples include improving skin appearance and/or feel by providing a smoother, more even appearance and/or feel; increasing the thickness of one or more layers of the skin; improving the elasticity or resiliency of the skin; improving the firmness of the skin; and reducing the oily, shiny, and/or dull appearance of skin, improving the hydration status or moisturization of the skin, improving the appearance of fine lines and/or wrinkles, improving skin exfoliation or desquamation, plumping the skin, improving skin barrier properties, improve skin tone, reducing the appearance of redness or skin blotches, and/or improving the brightness, radiancy, or translucency of skin; preventing damage to skin via antioxidant approaches, including UV A and UV B induced damage, preventing formation of comedomes, balancing the skin microbiome or preventing acne.

As used herein, “skin care active” means a compound or combination of compounds that, when applied to skin, provide an acute and/or chronic benefit to skin or a type of cell commonly found therein. Skin care actives may regulate and/or improve skin or its associated cells (e.g., improve skin elasticity, hydration, skin barrier function, and/or cell metabolism).

As used herein, “skin care composition” means a composition that includes a skin care active and regulates and/or improves skin condition.

As used herein, “substantially free” means less than 0.5%, less than 0.25%, less than 0.1%, less than 0.05%, and/or less than 0.01%.

Other than in the operating examples, or where otherwise indicated, all numbers expressing quantities of ingredients and/or reaction conditions are to be understood as being modified in all instances by the term “about” which can encompass ±10%, ±8%, ±6%, ±5%, ±4%, ±3%, ±2%, ±1%, or ±0.5%.

All numbers expressing pH values are to be understood as being modified by the term “about,” and as encompassing readings using a pH meter having a variation of up to ±10%, as up to ±9%, up to ±8%, up to ±7%, up to ±6%, up to ±5%, up to ±4%, up to ±3%, up to ±2%, or up to ±1%, which a skilled person will recognize relates to the inherent variation in pH meters.

All percentages are by weight of the cosmetic composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. Unless otherwise indicated, all measurements are understood to be made at approximately 21° C. and at ambient conditions, where “ambient conditions” means conditions under about 1 atmosphere of pressure and at about 50% relative humidity. All numeric ranges are inclusive of narrower ranges; delineated upper and lower range limits are interchangeable to create further ranges not explicitly delineated.

It is to be understood that, as used herein the terms “the,” “a,” or “an,” mean “at least one,” and should not be limited to “only one” unless explicitly indicated to the contrary.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a method that comprises A+B+C include embodiments where a method consists of A+B+C and embodiments where a method consists essentially of A+B+C. As described, the phrase “at least one of A, B, and C” is intended to include “at least one A or at least one B or at least one C,” and is also intended to include “at least one A and at least one B and at least one C.”

### Test Methods

### Microbial Susceptibility Testing (MST)

Fungal microbial susceptibility is tested as follows: Standardized ATCC strains of the yeast Candida albicans (ATCC #10231) and mold Aspergillus brasiliensis (ATCC #16404) are mixed in equal volumes such that the concentration of the fungal pool is approximately 7-9 log cfu/ml. This inoculum is added at a ratio of 1% v/w to product to deliver approximately 5-7 log cfu/g in product, and is incubated at 20-25° C. for up to 14 days. Organism survival is measured during and at the end of the incubation period by neutralizing an aliquot of inoculated sample in Modified Letheen Broth containing 1.5% polysorbate 80 and 1% Lecithin (MLBTL). Successive dilutions are transferred into petri dishes containing Modified Letheen Agar containing 1.5% polysorbate 80 (MLAT) and the agar plates are incubated at least 3 days at 30-35° C. Fungal colony forming units (cfus) are then enumerated, and a fungal log reduction from the starting log cfu/g challenge level is reported. Greater log cfu/g reduction values indicate greater anti-fungal robustness. A sample passes MST if, at any point during the shelf life, there is a reduction of fungal microbes by 2 logs or greater (rounded to the nearest integer) by 14 days post-inoculation.

### pH Test Method

pH can be using a standard pH meter such as, for example, a Beckman Coulter model PHI1410 pH meter equipped with a general-purpose probe (manufactured by Beckman Coulter, Brea, Calif., U.S.A.). The pH meter is calibrated according to the manufacturer's instructions. Measurements are performed after storing the compositions at room temperature (approximately 23° C.±2° C.) for approximately 24 hours.

### EXAMPLES

The following data and examples are provided to help illustrate the skincare compositions described herein. The exemplified compositions are given solely for the purpose of illustration and are not to be construed as limitations of the present disclosure, as many variations thereof are possible without departing from the spirit and scope of the disclosure. All parts, percentages, and ratios herein are by weight unless otherwise specified.

The Examples in the Tables, below, were prepared by first combining the water phase ingredients (water and glycerin) in a container and mixing until uniform. The dispersed phase ingredients (all other materials except water and glycerin) are combined in a separate container and mixed until uniform. Each of the phases are heated to ˜ 75° C. Finally, the dispersed phase is added to the water phase with high shear mixing (e.g., Rotor Stator high shear mixer) to form an emulsion. The emulsion is then cooled and continuously mixed until it reaches a temperature below ˜40° C., when, it is ready to be poured into a container for storage, use, or testing.

In Table 4 to Table 6, below, the MST Yeast/Mold was determined using the Microbial Susceptibility Test, described herein, and the appearance of the film (e.g., uniform or inconsistent and grainy?) was assessed according to the Drawdown Method, described herein. The Examples were considered acceptable if they passed MST and the film appeared smooth and uniform according to the Drawdown Method. If the sample did not pass MST, it was often not evaluated according to the Drawdown Method. Example 10 was only tested at the initial time point for yeast/mold and it is uncertain if this example would provide sufficient antimicrobial hostility and/or ZnO dispersion.

In Table 4 to Table 6, below, Examples 8, 9, 13, and 14 had a smooth, uniform film and had a log reduction of 2 or more, when rounded to the nearest integer, of yeast/mold according to MST at initial, after 1 month at 40° C., and 3 months at 40° C. This indicates that Examples 8, 9, 13, and 14 may be acceptable. It was surprising that these examples, which included a higher level of preservative system ingredients, as compared to the other examples, had better ZnO dispersion and were also stable emulsions. Example 16 may also be consumer acceptable. It had a smooth, uniform film and although MST was not performed at all three time points, the initial log reduction was 2.7 and it is likely that this composition would have acceptable yeast/mold hostility throughout the shelf life of the product.

TABLE 3
Chassis 1

| | Wt. % |
|---|---|
| Water | Q.S. |
| Tocopherol¹ (70% tocopherol in oil) | 0.02 (0.014) |
| Emollient | 3-5 |
| Coconut Alkanes (and) Coco-Caprylate/Caprate² | 5 |
| Cetearyl Alcohol (and) Coco-Glucoside³ | 4 |
| Arachidyl Alcohol (and) Behenyl Alcohol (and) Arachidyl | 1 |
| Glucoside⁴ | |
| Zinc Oxide (and) Caprylic/Capric Triglyceride (and) | 30.3 (20.00) |
| Polyhydroxystearic acid (66% ZnO)⁵ | |
| Glycerin | 3 |
| Xanthan Gum⁶ | 0.4 |

TABLE 4

| | Ex. 1 | Ex. 2 | Ex. 3 | Ex. 4 | Ex. 5 | Ex. 6 |
|---|---|---|---|---|---|---|
| | (Wt. %) | (Wt. %) | (Wt. %) | (Wt. %) | (Wt. %) | (Wt. %) |
| Chassis 1 | Q.S. | Q.S. | Q.S. | Q.S. | Q.S. | Q.S. |
| Benzyl Alcohol | | 0.9 | | | | |
| 1,2-Hexanediol | | | 0.2 | 0.4 | 0.8 | 0.68 |
| (and) 1,2- | | | | | | |
| Octanediol⁷ | | | | | | |
| Hydroxyacetophenone⁸ | | | | | | |
| Phenoxyethanol | | | | | | 0.5625 |
| Phenoxyethanol | | | | | | |
| (and) | | | | | | |
| Chlorphenesin | | | | | | |
| (and) Aqua | | | | | | |
| (and) Glycerin⁹ | | | | | | |

MST Yeast/Mold

| | | | | | | |
|---|---|---|---|---|---|---|
| Initial (Log | <1.1 | 2.9 | 1.2 | 1.1 | 1.1 | 2.2 |
| Reduction) | | | | | | |
| 1 Month at | | 0.5 | | | | 2.1 |
| 40° C. (Log | | | | | | |
| Reduction) | | | | | | |
| 3 Months at | | 0.4 | | | | 1.9 |
| 40° C. (Log | | | | | | |
| Reduction) | | | | | | |

Drawdown Film

| | | | | | | |
|---|---|---|---|---|---|---|
| Appearance | N/A | Uniform | N/A | N/A | N/A | Inconsistent |
| Following the | | Film, Pass | | | | Film, Grainy, |
| Drawdown | | | | | | Fail |
| Method | | | | | | |

Conclusion

| | | | | | | |
|---|---|---|---|---|---|---|
| Acceptable? | No | No | No | No | No | No |

TABLE 5

| | Ex. 7 | Ex. 8 | Ex. 9 | Ex. 10 | Ex. 11 |
|---|---|---|---|---|---|
| | (Wt. %) | (Wt. %) | (Wt. %) | (Wt. %) | (Wt. %) |
| Chassis 1 | Q.S. | Q.S. | Q.S. | Q.S. | Q.S. |
| Benzyl Alcohol | 0.4875 | | 0.585 | | |
| 1,2-Hexanediol (and) | 0.68 | 0.76 | 0.76 | 0.8 | |
| 1,2-Octanediol⁷ | | | | | |
| Hydroxyacetophenone⁸ | | | | | 0.15 |
| Phenoxyethanol | | 0.7125 | | 0.375 | 0.375 |
| Phenoxyethanol (and) | | | | | |
| Chlorphenesin (and) | | | | | |
| Aqua (and) Glycerin⁹ | | | | | |

MST Yeast/Mold

| | | | | | |
|---|---|---|---|---|---|
| Initial | 2.2 | 4 | 4 | 2.2 | 0.7 |
| (Log Reduction ≥2) | | | | | |
| 1 Month at 40° C. | 1.9 | 3.4 | 3.3 | | 0.7 |
| (Log Reduction) | | | | | |
| 3 Months at 40° C. | 1.1 | 3.1 | 3.1 | | 0.8 |
| (Log Reduction) | | | | | |

Drawdown Film

| | | | | | |
|---|---|---|---|---|---|
| Appearance Following | Inconsistent | Uniform | Uniform | N/A | Inconsistent |
| the Drawdown | Film, Grainy, | Film, Pass | Film, Pass | | Film, Grainy, |
| Method? | Fail | | | | Fail |

Conclusion

| | | | | | |
|---|---|---|---|---|---|
| Acceptable? | No | Yes | Yes | Unknown | No |

TABLE 6

| | Ex. 12 | Ex. 13 | Ex. 14 | Ex. 15 | Ex. 16 |
|---|---|---|---|---|---|
| | (Wt. %) | (Wt. %) | (Wt. %) | (Wt. %) | (Wt. %) |
| Chassis 1 | Q.S. | Q.S. | Q.S. | Q.S. | Q.S. |
| Benzyl Alcohol | 0.325 | | 0.39 | | |
| 1,2-Hexanediol (and) | | | | | |
| 1,2-Octanediol⁷ | | | | | |
| Hydroxyacetophenone⁸ | 0.15 | 0.3 | 0.3 | | |
| Phenoxyethanol | | 0.475 | | | |
| Phenoxyethanol (and) | | | | 0.6 | 1 |
| Chlorphenesin (and) | | | | | |
| Aqua (and) Glycerin⁹ | | | | | |

MST Yeast/Mold

| | | | | | |
|---|---|---|---|---|---|
| Initial | 0.8 | 3.4 | 3.1 | 1.1 | 2.7 |
| (Log Reduction) | | | | | |
| 1 Month at 40° C. | 0.8 | 3 | 3.1 | | |
| (Log Reduction ≥2) | | | | | |
| 3 Months at 40° C. | 0.8 | 3.2 | 2.9 | | |
| (Log Reduction) | | | | | |

Drawdown Film

| | | | | | |
|---|---|---|---|---|---|
| Appearance Following | Inconsistent | Uniform | Uniform | Uniform | Uniform |
| the Drawdown | Film, Grainy, | Film, Pass | Film, Pass | Film, Pass | Film, Pass |
| Method? | Fail | | | | |

Conclusion

| | | | | | |
|---|---|---|---|---|---|
| Acceptable? | No | Yes | Yes | No | Yes |

### Tradenames and Suppliers for Table 3 to Table 6:

1. Tocomix™ 70 IP from Advanced Organic Materials
2. Vegelight® 1214LC from Grant Industries®
3. Montanvo™ 82 from Seppic®
4. Montanov™ 202 from Seppic®
5. G CP66XZ dispersion from Kobo®
6. Keltrol® 1000 from CP Kelco®
7. SymDiol® 68 from Symrise®. SymDiol® contains from 25% to <50% of 1,2-Hexanediol and 1,2-Octanediol.
8. SymSave® H from Symrise®
9. Microcare® PHC from Thor®. Microcare® PHC contains >25% phenoxyethanol and 10-25% chlorphenesin.

An experiment was conducted to compare the ZnO dispersion in Example 13 (Table 6), which passed MST and had a uniform film, and Example 11 (Table 5), which did not pass MST had an inconsistent, grainy film. The examples were analyzed using at −103° C.

To perform the SEM analysis Carbon tape was applied to a copper stub. 0.1 mL sample was placed on the carbon tape using disposable Wiretol micropipette. A glass slide was slid down the carbon tape to produce and even thin film of product. Sample stub was plunged frozen in liquid nitrogen and placed on the Gatan Alto Pre-Prep sampler at −95° C. Scanning electron microscopy (SEM)-Hitachi FE-SEM SU8230 SN 1632-04 analysis conducted at −103° C. to minimize water loss during analysis. Sample coated with gold/palladium for 90 seconds at 11 mAu. Magnifications reported on images.

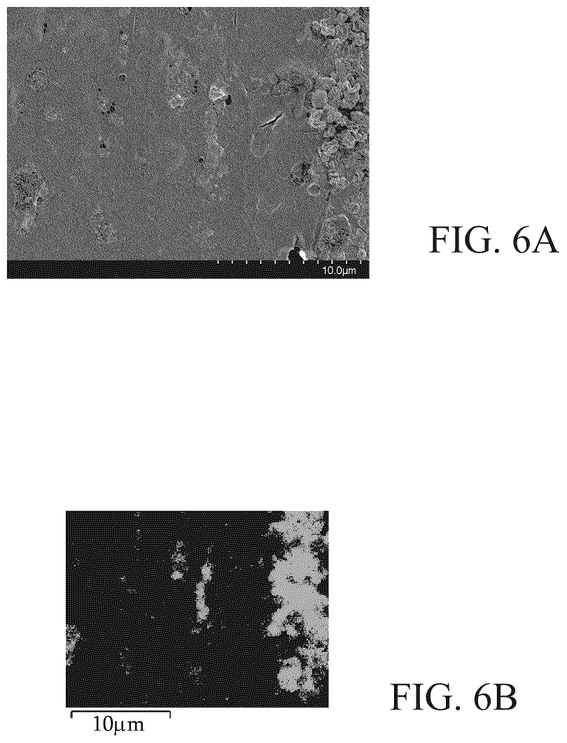

FIG. 6A is an SEM photograph of Example 11, taken at a magnification of 5000×. The photograph suggests that the ZnO in Example 11 tends to form agglomerates, resulting in the formation of larger distinct zones of agglomerates. agglomerates and forms larger zones of discrete agglomerates. FIG. 6B shows the energy dispersion of zinc, which reveals that the agglomerates are primarily concentrated in the zinc component.

FIG. 7A is an SEM photograph of Example 13, taken at a magnification of 5000×. FIG. 7A indicates that the ZnO in Example 13 is consistently dispersed throughout the composition, without forming significant agglomerates. This observation suggests a more uniform distribution of ZnO particles in Example 13 as compared to Example 11. In addition, FIG. 7B displays the energy dispersion of zinc, revealing that the zinc component exhibits significantly better dispersion in Example 13 as compared to Example 11.

### Combinations:

A. A skin care composition comprising:
   a. zinc oxide;
   b. from about 0.55% to about 2%, by weight of the composition, a preservative system comprising:
      i. at least 0.15%, by weight of the composition, 1,2-octanediol;
      ii. at least 0.5%, by weight of the composition, benzyl alcohol and/or at least 0.6%, by weight of the composition, phenoxyethanol;
   c. a dermatologically acceptable carrier.
B. The skin care composition according to Paragraph A, wherein the preservative system comprises from 0.15% to about to about 0.5%, by weight of the composition, 1,2-octanediol; preferably from about 0.18% to about 0.4%, by weight of the composition, 1,2-octanediol; and more preferably alternatively from about 0.18% to about 0.38%, by weight of the composition, 1,2-octanediol.
C. The skin care composition according to Paragraphs A-B, wherein the preservative system comprises 0.5% to about 1%, by weight of the composition, benzyl alcohol; preferably from about 0.5% to about 0.8%, by weight of the composition, benzyl alcohol; more preferably from about 0.5% to about 0.75%, by weight of the composition, benzyl alcohol; and even more preferably from about 0.55% to about 0.7% by weight of the composition, benzyl alcohol.
D. The skin care composition according to Paragraphs A-C, wherein the weight ratio of benzyl alcohol to 1,2-octanediol can be from about 1.4:1 to about 4:1, preferably from about 1.5:1 to about 3:1.
E. The skin care composition according to Paragraphs A-D, wherein the preservative system comprises from about 0.6% to about 1%, by weight of the composition, phenoxyethanol; preferably from about 0.65% to about 0.9%, by weight of the composition, phenoxyethanol; more preferably from about 0.67% to about 0.85%, by weight of the composition, phenoxyethanol; and even more preferably from about 0.7% to about 0.8%, by weight of the composition, phenoxyethanol.
F. The skin care composition according to Paragraphs A-E, wherein the weight ratio of phenoxyethanol to 1,2-octanediol is at least 1.5:1, preferably at least 2:1, more preferably at least 2.5:1, and even more preferably at least ≥3:1.
G. A skin care composition comprising:
   a. zinc oxide
   b. a preservative system comprising:
      i. at least 0.2%, by weight of the composition, hydroxyacetophenone;
      ii. at least 0.4%, by weight of the composition, phenoxyethanol and/or at least 0.2%, by weight of the composition, benzyl alcohol.
H. The skin care composition according to Paragraph G, wherein the preservative system comprises from about 0.2% to about 0.8%, by weight of the composition, hydroxyacetophenone; preferably from about 0.25% to about 0.6%, by weight of the composition, hydroxyacetophenone; more preferably from about 0.27% to about 0.5%, by weight of the composition, hydroxyacetophenone, and even more preferably from about 0.3% to about 0.4%, by weight of the composition, hydroxyacetophenone.
I. The skin care composition according to Paragraphs G-H, wherein the preservative system comprises from about 0.2% to about 0.8%, by weight of the composition, phenoxyethanol; preferably from about 0.3% to about 0.7%, %, by weight of the composition, phenoxyethanol; more preferably from about 0.35% to about 0.65%, %, by weight of the composition, phenoxyethanol; and even more preferably from about 0.43% to about 0.55%, %, by weight of the composition, phenoxyethanol.
J. The skin care composition according to Paragraphs G-I, wherein the preservative system comprises from about 0.2% to about 0.6%, by weight of the composition, benzyl alcohol; preferably from about 0.3% to about 0.55%, by weight of the composition, benzyl alcohol; more preferably from about 0.3% to about 0.5%, by weight of the composition, benzyl alcohol; and even more preferably from about 0.35% to about 0.45%, by weight of the composition, benzyl alcohol.
K. A skin care composition comprising:
   a. zinc oxide;
   b. a preservative system comprising:
      i. from about 0.07% to about 0.7%, by weight of the composition, chlorphenesin; preferably from about 0.1% to about 0.5%, by weight of the composition, chlorphenesin; more preferably from about 0.15% to about 0.4%, by weight of the composition, chlorphenesin; and even more preferably from about 0.2% to about 0.3%, by weight of the composition, chlorphenesin; and
      ii. from about 0.16% to about 1%, by weight of the composition, phenoxyethanol; preferably from about 0.2% to about 0.9%, by weight of the composition, phenoxyethanol; more preferably from about 0.3% to about 0.8%, by weight of the composition, phenoxyethanol; and even more preferably from about 0.4% to about 0.7%, by weight of the composition, phenoxyethanol;
   c. a dermatologically acceptable carrier.
L. The skin care composition according to Paragraphs A-K, wherein the composition is an emulsion.
M. The skin care composition according to Paragraphs A-L, wherein the composition comprises a log reduction of ≥2 for yeast and mold, according to the Microbial Susceptibility Testing.
N. The skin care composition according to Paragraphs A-M, wherein the zinc oxide is dispersed.
O. A skin care composition comprising:
   a. zinc oxide; wherein the zinc oxide is dispersed;
   b. from about 0.6% to about 1.5%, by weight of the composition, of a preservative system; preferably from about 0.67% to about 1.25%, by weight of the composition, of a preservative system; and even more preferably from about 0.69% to about 1.1%, by weight of the composition, of a preservative system; wherein the preservative system comprises:
      i. a registered preservative material chosen from phenoxyethanol, benzyl alcohol, chlorphenesin, or mixtures thereof;
      ii. optionally a booster chosen from hydroxyacetophenone, 1,2-octanediol, or mixtures thereof;
   c. a dermatologically acceptable carrier,
      wherein the composition comprises a log reduction of ≥2 for yeast and mold, according to the Microbial Susceptibility Testing;
      wherein the skin care composition is an emulsion.
P. The skin care composition of according to Paragraph O, wherein the composition comprises a uniform film, according to the Drawdown Method.
Q. The skin care composition according to Paragraphs O-P, wherein the preservative system comprises at least 0.25%, by weight of the composition, phenoxyethanol; preferably at least 0.35%, by weight of the composition, phenoxyethanol; more preferably at least 0.4%, by weight of the composition, phenoxyethanol; and even more preferably at least 0.5%, by weight of the composition, phenoxyethanol.
R. The skin care composition according to Paragraphs O-Q, wherein the preservative system comprises at least 0.5%, by weight of the composition, benzyl alcohol; and preferably at least 0.55%, by weight of the composition, benzyl alcohol.
S. The skin care composition according to Paragraphs O-R, wherein the preservative system comprises at least 0.16%, by weight of the composition, hydroxyacetophenone; preferably at least 0.2%, by weight of the composition, hydroxyacetophenone; more preferably at least 0.25%, by weight of the composition, hydroxyacetophenone; and even more preferably at least 0.30%, by weight of the composition, hydroxyacetophenone.
T. The skin care composition according to Paragraphs O-S, wherein the preservative system comprises at least 0.15%, by weight of the composition, 1,2-octanediol; preferably at least 0.19%, by weight of the composition, 1,2-octanediol; more preferably at least 0.25%, and even more preferably at least 0.30%, by weight of the composition, 1,2-octanediol.
U. The skin care composition according to Paragraphs O-T, wherein the preservative system comprises at least 0.07%, by weight of the composition, chlorphenesin; preferably at least 0.1%, by weight of the composition, chlorphenesin; more preferably at least 0.15%, by weight of the composition, chlorphenesin; and even more preferably at least 0.20%, by weight of the composition, chlorphenesin.
V. The skin care composition according to Paragraphs A-U, wherein the pH is at least 7, preferably from about 7.1 to about 8.2.
W. The skin care composition according to Paragraphs A-V, wherein the skin care composition comprises a broad spectrum SPF of at least 15, preferably at least 25, and more preferably at least 30.
X. The skin care composition according to Paragraphs A-W, wherein the composition comprises from about 7% to about 25%, by weight of the composition, zinc oxide; preferably from about 11% to about 25%, by weight of the composition, zinc oxide; more preferably from about 15% to about 25%, by weight of the composition, zinc oxide.
Y. The skin care composition according to Paragraphs A-X, further comprising titanium dioxide.
Z. The skin care composition according to Paragraphs A-Y, wherein the composition is formulated without or free of chemical sunscreen actives chosen from aminobenzoic acid, avobenzone, cinoxate, dioxybenzone, ensulizole, homosalate, meradimatem octinoxate, octisalate, octocrylene, oxybenzone, padimate, sulisobenzone, trolamine salicylate, or mixtures thereof.
AA. The skin care composition according to Paragraphs A-Z, wherein the skin care composition is an emulsion.
BB. The skin care composition according to Paragraphs AA, wherein the emulsion is physically stable, wherein there is no phase separation by visual detection.
CC. The skin care composition according to Paragraphs A-Z, wherein the composition is formulated without or free of benzoic acid.
DD. The skin care composition according to Paragraphs A-AA, wherein the composition is formulated without or free of organic acids.
EE. The skin care composition according to Paragraphs A-BB, wherein the composition is free of DMDM hydantoin, imadazolidinyl urea, diazlidinyl urea, sodium hydroxyl, and methyl glycinate.
FF. The skin care composition according to Paragraphs A-CC, wherein the composition is formulated without or free of phenylpropanol, ethylhexylglycerin, methylheptylglycerin, and iodopropynyl butyl carbamate.
GG. The skin care composition according to Paragraphs A-DD, wherein the weight ratio of zinc oxide to preservative system can be from about 10:1 to about 40:1, preferably from about 15:1 to about 35:1, more preferably from about 18:1 to about 30:1, and even more preferably from about 18:1 to about 26:1.
HH. Use of a preservative system in a skin care composition of according to Paragraphs A-E for providing a physically stable composition, wherein the preservative system comprises:
   a. at least 0.15%, by weight of the composition, 1,2-octanediol;
   b. at least 0.5%, by weight of the composition, benzyl alcohol and/or at least 0.6%, by weight of the composition, phenoxyethanol.
II. Use of a preservative system in a skin care composition according to Paragraphs G-I, for providing a physically stable composition, wherein the preservative system comprises:
   a. at least 0.2%, by weight of the composition, hydroxyacetophenone;
   b. at least 0.4%, by weight of the composition, phenoxyethanol and/or at least 0.2%, by weight of the composition, benzyl alcohol.
JJ. Use of a preservative system in a skin care composition according to Paragraphs O-U, for providing a physically stable composition, wherein the preservative system comprises:
   a. a registered preservative material chosen from phenoxyethanol, benzyl alcohol, chlorphenesin, or mixtures thereof;
   b. optionally a booster chosen from 1,2-octanediol, hydroxyacetophenone, or mixtures thereof.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A skin care composition comprising:
a. zinc oxide;
b. from about 0.55% to about 2%, by weight of the composition, a preservative system comprising:
i. at least 0.15%, by weight of the composition, 1,2-octanediol;
ii. at least 0.5%, by weight of the composition, benzyl alcohol and/or at least 0.6%, by weight of the composition, phenoxyethanol;
c. a dermatologically acceptable carrier.

2. The skin care composition of claim 1, wherein the composition is an emulsion, and the emulsion is physically stable.

3. The skin care composition of claim 1, wherein the composition comprises a log reduction of ≥2 for yeast and mold, according to the Microbial Susceptibility Testing.

4. The skin care composition of claim 3, wherein the zinc oxide is dispersed throughout the skin care composition.

5. The skin care composition of claim 1, wherein the pH is at least 7.

6. The skin care composition of claim 1, wherein the skin care composition comprises a broad spectrum SPF of at least 15.

7. The skin care composition of claim 1, wherein the composition comprises from about 7% to about 25%, by weight of the composition, zinc oxide.

8. The skin care composition of claim 1, wherein the preservative system comprises from 0.15% to about to about 0.5%, by weight of the composition, 1,2-octanediol and from 0.5% to about 1%, by weight of the composition, benzyl alcohol.

9. The skin care composition of claim 1, wherein the preservative system comprises from 0.15% to about to about 0.5%, by weight of the composition, 1,2-octanediol and from about 0.6% to about 1%, by weight of the composition, phenoxyethanol.

10. A skin care composition comprising:
a. zinc oxide; wherein the zinc oxide is dispersed;
b. from about 0.6% to about 1.5%, by weight of the composition, of a preservative system comprising:
i. a registered preservative material chosen from phenoxyethanol, benzyl alcohol, chlorphenesin, or mixtures thereof;
ii. optionally a booster chosen from 1,2-octanediol, hydroxyacetophenone, or mixtures thereof;
c. a dermatologically acceptable carrier,
wherein the composition comprises a log reduction of ≥2 for yeast and mold, according to the Microbial Susceptibility Testing;
wherein the skin care composition is an emulsion and the emulsion is stable.

11. The composition of claim 10, wherein the composition comprises a uniform film, according to the Drawdown Method.

12. The skin care composition of claim 10, wherein the composition is formulated without chemical sunscreen actives.

13. The skin care composition of claim 10, wherein the composition is formulated without benzoic acid.

14. The skin care composition of claim 10, wherein the composition is formulated without organic acids.

15. The skin care composition of claim 10, wherein the composition is formulated without DMDM hydantoin, imadazolidinyl urea, diazlidinyl urea, sodium hydroxyl, and methyl glycinate.

16. The skin care composition of claim 10, wherein the composition is formulated without phenylpropanol, ethylhexylglycerin, methylheptylglycerin, and iodopropynyl butyl carbamate.

17. A skin care composition comprising:
a. zinc oxide;
b. a preservative system comprising:
i. at least 0.2%, by weight of the composition, hydroxyacetophenone;
ii. at least 0.4%, by weight of the composition, phenoxyethanol and/or at least 0.2%, by weight of the composition, benzyl alcohol.

18. The skin care composition of claim 17, wherein the preservative system comprises from about 0.2% to about 0.8%, by weight of the composition, hydroxyacetophenone.

19. The skin care composition of claim 18, wherein the preservative system comprises from about 0.4% to about 0.8%, by weight of the composition, phenoxyethanol.

20. The skin care composition of claim 18, wherein the preservative system comprises from about 0.2% to about 0.6%, by weight of the composition, benzyl alcohol.
